(19) United States
(12) Patent Application Publication
(10) Pub. No.: US 2024/0001000 A1
(43) Pub. Date: Jan. 4, 2024

(54) COMPOSITIONS AND METHODS PROMOTING GROWTH OF PERIPHERAL NERVOUS TISSUE

(71) Applicant: MAYO FOUNDATION FOR MEDICAL EDUCATION AND RESEARCH, Rochester, MN (US)

(72) Inventors: Atta Behfar, Rochester, MN (US); Peter C. Amadio, Rochester, MN (US)

(21) Appl. No.: 18/248,615

(22) PCT Filed: Oct. 12, 2021
(86) PCT No.: PCT/US2021/054547

§ 371 Date: Apr. 11, 2023

Related U.S. Application Data
(60) Provisional application No. 63/234,567, filed on Aug. 18, 2021.
(60) Provisional application No. 63/091,240, filed on Oct. 13, 2020.

(51) Int. Cl. A61L 27/36

(52) U.S. Cl. CPC A61L 27/3675; A61L 2430/32; A61L 27/3616

(57) ABSTRACT

A composition includes Purified Exosome Product (PEP) and a pharmaceutically acceptable carrier that includes a surgical glue or tissue adhesive. In some embodiments, the PEP includes spherical or spheroid exosomes having a diameter no greater than 300 nm. In some embodiments, the PEP includes from 1% to 20% CD63⁻ exosomes and from 80% to 99% CD63⁺ exosomes. In some embodiments, the PEP includes at least 50% CD63⁻ exosomes. The composition may be applied to injured peripheral nervous tissue to promote growth of peripheral nervous tissue and/or treat the injured peripheral nervous tissue. The peripheral nervous tissue may be autologous or may be allogeneic.

### CROSS-REFERENCE TO RELATED APPLICATION

This application claims the benefit of U.S. Provisional Patent Application No. 63/091,240, filed Oct. 13, 2020, and U.S. Provisional Patent Application No. 63/234,567, filed Aug. 18, 2021, each of which is incorporated herein by reference in its entirety.

### SUMMARY

This disclosure describes, in one aspect, a composition that includes Purified Exosome Product (PEP) and a pharmaceutically acceptable carrier that includes a surgical glue or tissue adhesive.

In some embodiments, the PEP includes spherical or spheroid exosomes having a diameter no greater than 300 nm. In some of these embodiments, the spherical or spheroid exosomes have a mean diameter of 110 nm±90 nm. In some of these embodiments, the spherical or spheroid exosomes have a mean diameter of 110 nm±30 nm.

In some embodiments, the PEP includes from 1% to 20% CD63⁻ exosomes and from 80% to 99% CD63⁺ exosomes.

In some embodiments, the PEP includes at least 50% CD63⁻ exosomes.

In another aspect, this disclosure describes a method of promoting growth of peripheral nervous tissue. Generally, the method includes the applying to injured peripheral nervous tissue any embodiment of a composition that includes PEP and a pharmaceutically acceptable carrier that includes a surgical glue or a tissue adhesive.

In some embodiments, the peripheral nervous tissue is an autograft. In some embodiments, the peripheral nervous tissue is an allograft.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Intraoperative findings. Group I: panel A, panel B, panel C. Group II: panel D, panel E, panel F. Group III: panel G, panel H, panel I. Immediately after autograft and local administration of glue: Panel A, panel D, panel G. Three days post-surgery: panel B, panel E, panel, H. Seven days post-surgery: panel C, panel F, panel I. Scale bare=10 mm.

FIG. 2. The mRNA expression of GAP43 and S100b as measured by pRT-PCR taken from the three groups. (A) mRNA expression of GAP43 in the sciatic nerve. (B) mRNA expression of GAP43 in dorsal root ganglion. (C) mRNA expression of S100b in the sciatic nerve. (D) mRNA expression of S100b in dorsal root ganglion. The mRNA expression was calculated by the 2^{-ΔCt} method using GAPDH and beta actin as the housekeeping gene. The relative mRNA levels were represented as the ratios by comparing the expression of each group with that of Group I. n=4 for each group, *p<0.05 versus Group I; #p<0.05 versus Group II.

FIG. 3. Compound muscle action potential (CMAP), isometric tetric force (ITF), and muscle wet weight (MWW) measured at 12 weeks and 16 weeks in all three groups. (A) CMAP; (B) ITF; (C) MWW. Results are expressed as a percentage of the normal, contralateral side and are given as the mean and standard error. *p<0.05 between groups by one-way ANOVA.

FIG. 4. Transverse sections of peroneal nerve from all three groups. (A) Group I, 20× magnification. (B) Group II, 20× magnification. (C) Group III, 20× magnification. (D) Group I, 400× magnification. (E) Group II, 400× magnification. (F) Group III, 400× magnification. The size of regenerative axon in Group III is larger than in Group I and Group II.

FIG. 5. Axon measurements at 12 weeks and at 16 weeks in all three groups. (A) Axon density. (B) Axon diameter. Results are expressed as mean and standard error. *p<0.05 versus Group I; #p<0.05 versus Group II.

FIG. 6. Study design. The fabrication of PEP-fibrin glue-allograft and evaluations of nerve regeneration.

FIG. 7. In vivo characterization of PEP-fibrin glue allograft. (A) Scanning electron micrographs of allograft either without PEP (Allograft) or loaded with or without PEP (Allograft+PEP). The scale bars=5 μm and 1 μm. (B) NanoSight report presented absolute particles number (left) and size distribution (right). (C) Quantitative analysis of PEP releasing loaded in fibrin glue.

FIG. 8. In vivo characterization of PEP-fibrin glue allograft. Confocal micrographs showed uptake of PEP after immunostaining with CM-Dil, S100b, and Hoechst. The scale bar=5 μm.

FIG. 9. In vitro Schwann cell tests. (A) The analysis of cell proliferation of different PEP doses was measured by CCK8 assay. (B) Apoptosis rate of Schwann cells was evaluated using PI/annexin V-FITC staining and statistical results of early apoptosis rate. (C) Exemplary results of the wound healing dynamics based on scraper-wounded cell monolayer with treatment of different PEP doses after 36 hours culturing by IncuCyte Live Cell Analysis System. (D) The closure of scratch area was analyzed by measuring the wound size compared to the initial wound size as 100%. *p<0.05.

FIG. 10. Gross observation of surgical nerve grafts at surgery, 12 weeks, and 16 weeks.

FIG. 11. Functional tests. (A) Compound muscle action potential (CMAP). (B) Isometric tetanic force. (C) Muscle weight of the tibialis anterior. (D) Ankle contracture angle. Tests were performed for the three treatment groups at 12 weeks and 16 weeks post-surgery. All the data were normalized to the healthy side. The analysis was conducted among the three test groups at two different time points. *p<0.05, **p<0.01, and ***p<0.001.

FIG. 12. Photographs of tibialis muscle at 12 weeks and 16 weeks after surgery.

FIG. 13. Morphology of the myelin sheath located in various cross sections of regenerated nerves. Photographs of ultrathin section of regenerated axon in rat peroneal nerve with toluidine blue staining for three experimental groups at 12 weeks and 16 weeks after surgery.

FIG. 14. Morphology of the myelin sheath located in various cross sections of regenerated nerves. (A) Density of axons. (B) Fascicular area. (C) Myelin thickness. Measurements were quantified using ImageJ software. The analysis was conducted among the three test groups and two different time points, 12 weeks, and 16 weeks after surgery. *p<0.05, **p<0.01, ***p<0.001.

FIG. 15. Histological and immunohistochemical staining of neural markers in regenerated nerves 12 weeks and 16 weeks after surgery. (A) Hematoxylin and eosin (H&E) staining results of the nerve graft. (B) Masson trichrome staining results of the nerve graft. (C) Immunohistochemical staining of S100b staining results of the nerve graft. (D) Immunohistochemical staining of NFH-N52 staining results of the nerve graft.

FIG. 16. Results of RT-PCR and histology. (A) The expression level of GAP43, GFAP, CNTF, and S100b mRNA in three groups at seven days following surgery. Normal nerves as control. (B) The expression of S100b and the percentage of S100b positive area was analyzed. (C) The expression of NFH-N52 staining, number of NFH-N52 positive cells was analyzed. *p<0.05, **p<0.01, ***p<0.001.

### DETAILED DESCRIPTION OF ILLUSTRATIVE EMBODIMENTS

This disclosure describes compositions and methods for improving repair of peripheral nervous system tissues. Generally, the composition includes a purified exosome product (PEP) that is applied to tissue of the peripheral nervous system. The peripheral nervous tissue can be autologous (e.g., an autograft) or may be allogeneic (e.g., an allograft).

PEP is a purified exosome product prepared using a cryodesiccation step that produces a product having a structure that is distinct from exosomes prepared using conventional methods. For example, PEP typically has a spherical or spheroidal structure rather than a crystalline structure. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation-naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁺ exosomes to CD63 exosomes in a PEP product by removing CD63⁺ exosomes from the naturally-isolated PEP preparation, then adding back a desired amount of CD63⁺ exosomes. In some embodiments, a PEP preparation can have only CD63⁻ exosomes.

In some embodiments, a PEP preparation can have both CD63⁺ exosomes and CD63-exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in various embodiments, the ratio of CD63⁺ exosomes to CD63⁻ exosomes in a PEP preparation may be 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, or 30:1. In certain embodiments, the PEP product is formulated to contain a 9:1 ratio of CD63⁺ exosomes to CD63⁻ exosomes.

PEP is fully characterized and methods for preparing PEP are described in International Patent Application No. PCT/US2018/065627 (published as International Publication No. WO 2019/118817), which is incorporated by reference herein in its entirety.

### Nerve Regeneration in an Autograft Model

The nerve autograft remains the gold standard when reconstructing peripheral nerve defects. Yet, while autograft repair can result in useful functional recovery, poor outcomes are common, and better treatments are needed. This disclosure describes the effect of purified exosome product (PEP) on functional motor recovery and nerve related gene expression. Local administration of PEP improved peripheral nerve regeneration profiles in a rat sciatic nerve reverse autograft model. Thus, PEP provides beneficial effects on peripheral nerve regeneration, gene profiles, and/or motor outcomes.

PEP manufactured under current good manufacturing practice (CGMP) conditions was evaluated for concentration and size homogeneity, documenting approximately 6-7×10¹² exosomes per ml with a size distribution of 120 nm to 150 nm. Proteomic profiling of PEP versus conditioned media collected from adipose-derived mesenchymal stem cells (AMSC) revealed altered protein profiles, which correlated with cell proliferation, negative regulation of cell apoptosis and neuroregenerative pathway activation. Cytoprotective biopotency was assessed using a human iPS-derived mixed neuronal culture system. Stimulation with the free radical generator LY83583 resulted in significant cell death in the control, as evidenced by caspase 3/7 activation, whereas neurons treated with 5% PEP exhibited a reduced level of apoptosis. Taken together, these data support the use of PEP as a therapeutic tool to accelerate healing of peripheral nerve grafts.

An in vivo rat model was used to evaluate the effects of PEP on functional motor recovery and nerve related gene expression. Of 96 rats, six rats were excluded from analysis. Three animals died under anesthesia. Three rats were euthanized because of a wound infection caused by autotomy. The remaining 90 rats were divided into three groups. 31 rats were placed in Group I (12 weeks=12, 16 weeks=11), 28 rats were placed in Group II (12 weeks=10, 16 weeks=10), and 31 rats were placed in Group III (12 weeks=11, 16 weeks=12) were included in the final analyses.

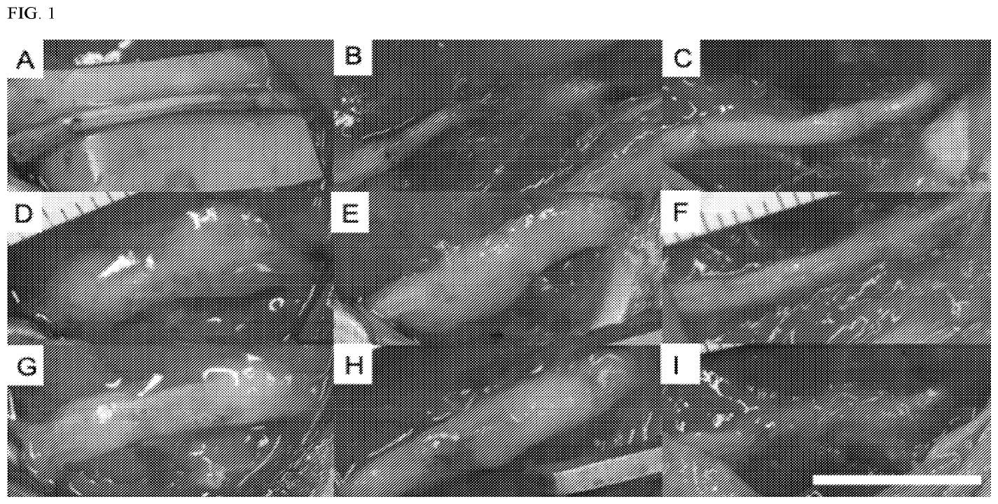

All grafts remained in continuity. Adhesion around the graft was observed in all groups. The fibrin glue in rats of Group II and rats of Group III was intact on day 3, and partly resorbed in rats of Group II and rats of Group III on day 7. There were no observable macroscopic differences between Group II and Group III at any time point (FIG. 1).

There were no significant differences in compound muscle action potential (CMAP) between groups at either 12 weeks or 16 weeks post-surgery. All groups significantly improved from 12 weeks to 16 weeks post-surgery (FIG. 3A).

There was a significant difference in maximum isometric tetanic force between groups at 16 weeks, whereas no difference was observed at 12 weeks. The tetanic force in Group III was significantly increased compared to Group I at 16 weeks. There was a significant difference between tetanic forces at both 12 weeks and at 16 weeks between Groups II and Group III, while no significant recovery in 12 weeks and 16 weeks post-surgery was observed in Group I (FIG. 3B).

There were no significant differences in muscle weight between groups at 12 weeks and 16 weeks post-surgery (FIG. 3C).

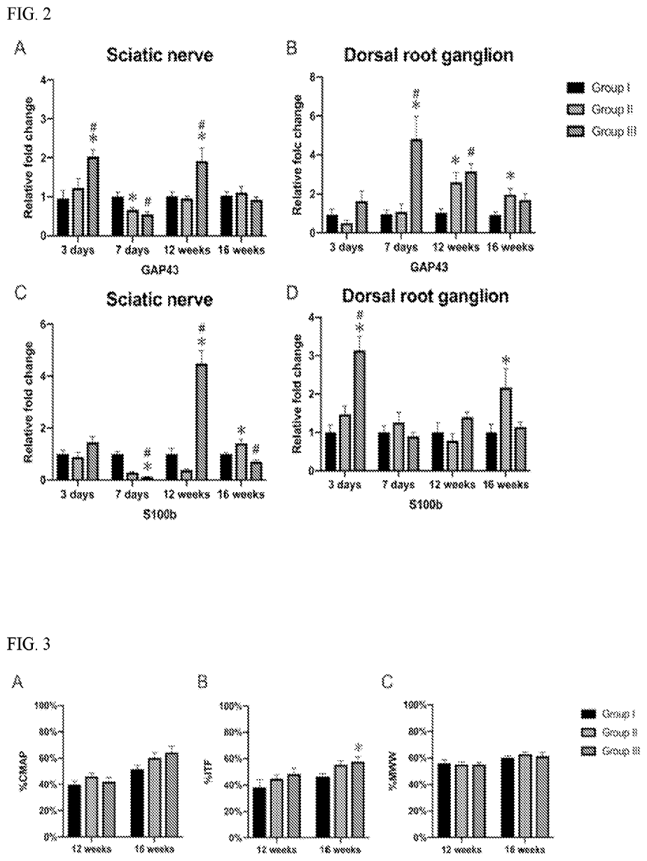

In the sciatic nerve, GAP43 gene expression was significantly upregulated in Group III compared with Group I and Group II at three days and 12 weeks post-surgery and downregulated compared with Group I at seven days post-surgery (FIG. 2A). S100b gene expression was significantly upregulated in Group III compared with Group I and Group II at 12 weeks post-surgery and downregulated at seven days and 16 weeks post-surgery (FIG. 2C).

In the dorsal root ganglion, GAP43 gene expression was significantly upregulated in Group III compared with Group I at all time points (FIG. 2B). S100b gene expression was significantly upregulated in Group III compared with Group I and Group II at three days post-surgery but there was no difference between Group I and Group III at other time points (FIG. 2D).

GAP43 is a nervous tissue-specific cytoplasmic protein produced by developing neurons in the central nervous system and by Schwann cells in the peripheral nervous system. S100b is secreted from activated Schwann cells and stimulates recruitment of Schwann cells and macrophages to the injury site to aid in axonal regeneration.

Hey1 gene expression was significantly upregulated in Group III at three days post-surgery. Sod1, Ntn1, Ptn, Slit2, Hes1, S100b, and Nrcam gene expression was significantly downregulated in Group III at seven days post-surgery. No significant difference in fold regulation was observed at dorsal root ganglion both three days and seven days post-surgery.

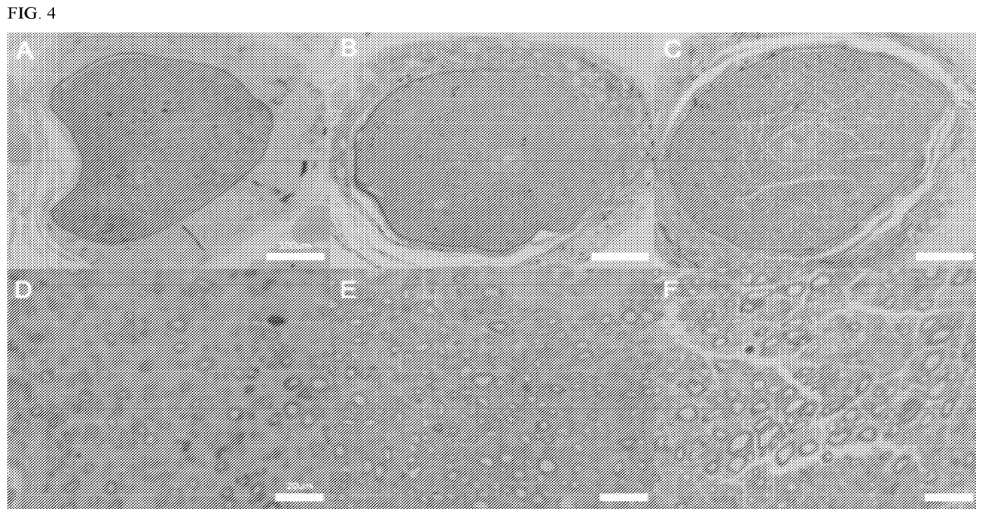

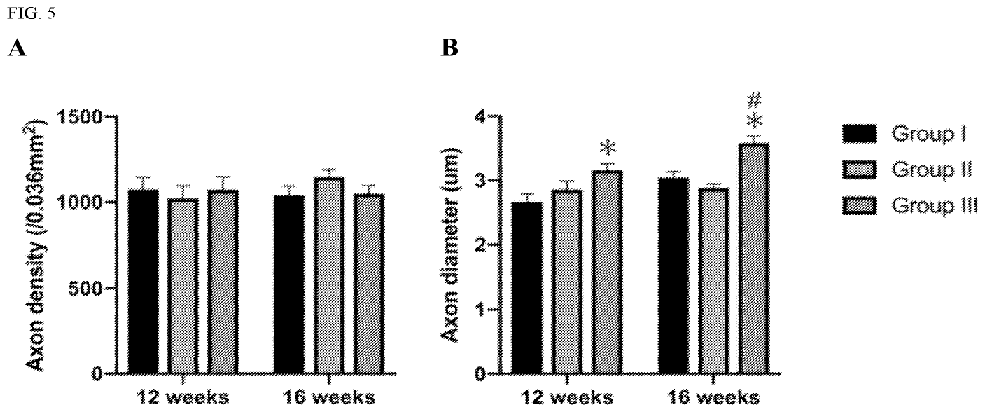

Axon diameter was significantly larger in Group III compared with Group I at 12 weeks post-surgery and significantly larger than Group I and Group II at 16 weeks post-surgery (FIG. 4A-C). No significant differences of axon density were observed between groups at 12 weeks and 16 weeks post-surgery (FIG. 4D-F).

Overall, the study revealed that the PEP-treated group (Group III) showed improvement in motor functional recovery and axon maturation compared to untreated grafts and TISSEEL vehicle controls. Furthermore, PEP regulated nerve regeneration-related gene expression.

The role and potential of exosomes derived from neurons in peripheral nerve regeneration are well established. For example, Schwann-cell-derived exosomes and their genetic cargo are likely involved in the process of Wallerian degeneration and nerve regeneration. Schwann-cell-derived exosomes and their miRNAs can accelerate the proliferation and myelination of Schwann cells, coordinate axonal growth, and enhance neurite outgrowth. Macrophage-derived exosomes can promote nerve regeneration and enhance migration and proliferation of Schwann cells.

While exosomes can be obtained from any cell type, whole blood is readily obtained and contains a variety of cell types known to participate in wound healing. PEP exosomes may be prepared from various non-neuronal cell types. (International Patent Application No. PCT/US2018/065627, published as International Publication No. WO 2019/118817). While exosomes of various cell origins are known, conventional methods for preparing exosomes can involve specialized and sometimes technically challenging cell harvest and, in some cases, technically challenging cell culture. In contrast, PEP, which uses a leukocyte-depleted pooled source, does not require antecedent cell culture. Furthermore, PEP may be stored at room temperature for a year or more, which facilitates the practicality of using PEP in the surgical setting.

In this study, the maximal isometric tetanic force of the tibialis anterior muscles at 16 weeks post-surgery was significantly improved by local administration of PEP in a rat peripheral nerve autograft model, whereas no significant difference was observed at 12 weeks post-surgery. This measure has advantages over walking track data in assessing the outcome of experimental nerve surgery, since it is not affected by postoperative complications such as joint contracture and autotomy. Outcomes were measured at both 12 weeks post-surgery and 16 weeks post-surgery because previous work has shown that, while 12 weeks is sufficient to assess the outcome after nerve repair or crush injury, longer follow up is needed to assess the results of nerve grafting.

The average axon diameter at 12 weeks post-surgery and 16 weeks post-surgery was significantly improved by local administration of PEP in a rat nerve autograft model, whereas no significant difference in axon density was observed at either 12 weeks post-surgery or 16 weeks post-surgery. Axon diameter and myelin thickness reflect nerve maturation, so these data suggest that the PEP accelerates maturation without increasing the number of axons crossing the two suture lines. Thus, local administration of a pro-vasculogenic and pro-mitotic exosome product, PEP, improved the nerve regeneration profiles in this reversed sciatic nerve autograft rat model, which suggest utility in a surgical setting.

### Nerve Regeneration in an Allograft Model

This disclosure also describes comparing autograft nerve regeneration with peripheral nerve regeneration using a combination of a plasma-derived purified exosome product (PEP) with decellularized allograft nerve. A fibrin glue was used as a carrier to deliver exosomes to the allograft. The exosome-fibrin glue mixture showed a steady releasing profile, and exosomes were readily taken up by Schwann cells (SCs), resulting in improved Schwann cell viability and migration in vitro. In animal model testing, the PEP-treated allograft yielded axonal regeneration, remyelination, and motor function recovery comparable to nerve autograft. The findings demonstrate that a decellularized nerve allograft treated with PEP increases neuro-regenerative gene expression and improves peripheral nerve function compared to a nerve allograft. This approach can promote and accelerate nerve regeneration within allografts, creating results in mixed nerves that are comparable to the nerve autograft.

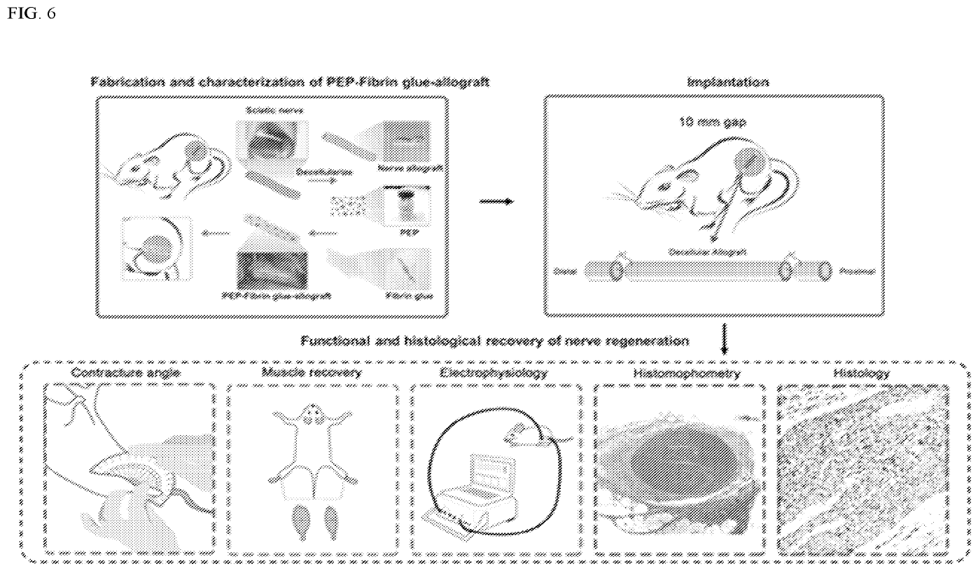

FIG. 6 illustrates the study design for evaluating PEP in the allograft model. Treatment of an allograft with PEP−TISSEEL was compared to allograft without PEP−TISSEEL and an autograft without PEP−TISSEEL treatment. FIG. 10 shows that wounds in all rats showed satisfactory healing without obvious formation of scar tissue at proximal or distal connections. No visible signs of inflammation, gap formation, seroma, or neuroma formation occurred in rats in any of the three groups.

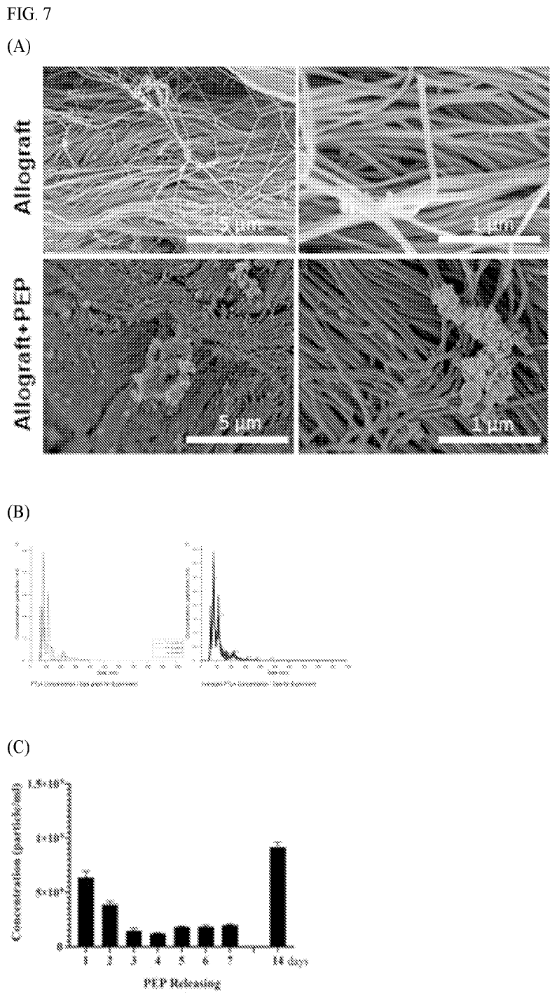

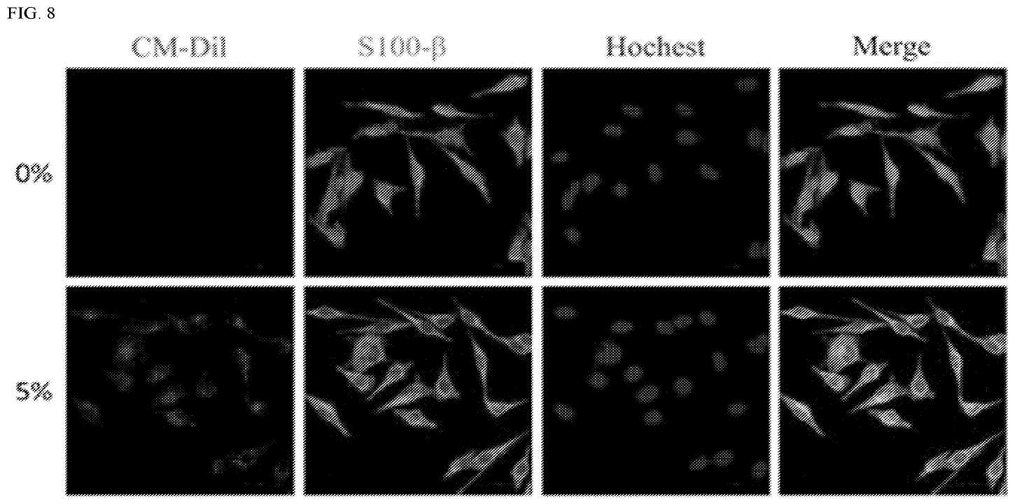

The release of PEP particles from the TISSEEL fibrin glue (Baxter International Inc., Deerfield, Illinois) is shown in FIG. 7. FIG. 7B shows the release profile of 100 nm PEP exosome particles. FIG. 7C shows that the PEP particles were gradually released from the fibrin glue and achieved a steady release rate after two days, providing a stable microenvironment for nerve regeneration. Confocal laser scanning microscopy was employed to detect the uptake of PEP in Schwann cells (FIG. 8). As revealed by immunostaining with antibodies against CM-dil and S100b, the released PEP was endocytosed by Schwann cells. The merged image showed the presence of PEP exosomes in Schwann cells. The surface of the allograft after co-culture with PEP was shown by scanning electron microscopy (FIG. 7A). Upon decellularization, allografts were processed to keep the original microstructure without any cells. Compared to the group treated with allograft alone (Allograft), clusters of PEP were deposited on the nerve fibers in the group treated with allograft and PEP (Allograft+PEP).

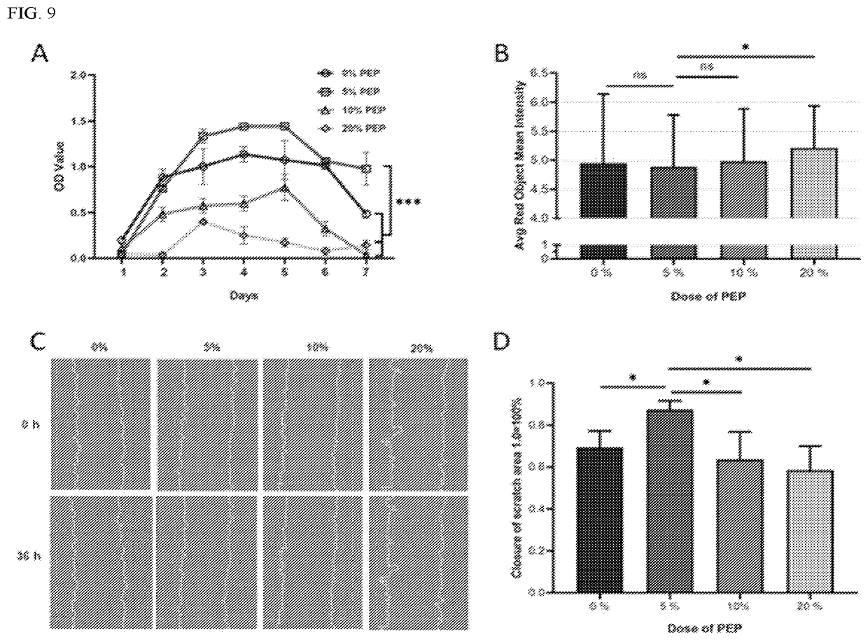

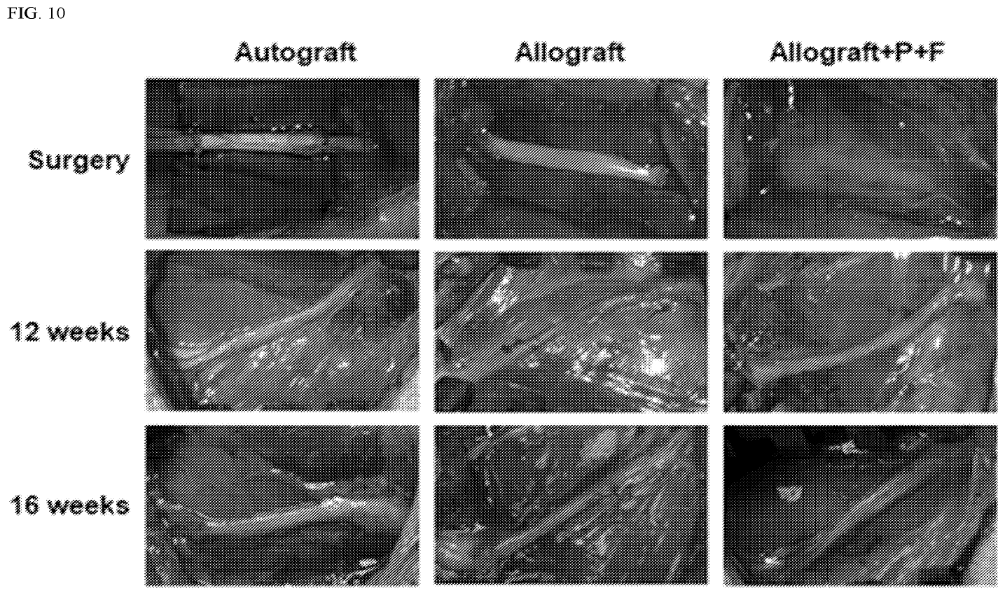

Schwann cells are involved in myelin formation and maintenance in the peripheral nervous system. As seen in FIG. 9, PEP significantly enhanced the proliferation of Schwann cells compared to the non-PEP group. Treatment with 5% PEP showed the most improved Schwann cell growth and wound healing rates (FIG. 9A). Schwann cells co-cultured with PEP grow faster through upregulating the capacity of cell proliferation and migration while downregulating the process of apoptosis.

To assess the general function of peripheral nerve regeneration, angle of ankle's contracture, CMAP, maximum isometric tetanic force, and weight of tibialis anterior muscle were evaluated at 12 weeks and 16 weeks after operations. The details of data are shown in Table 1.

TABLE 1
Summary of testing data

| | | | |
|---|---|---|---|
| Procedure | Autograft | Allograft | Allograft + PEP |

12 Weeks

| | | | |
|---|---|---|---|
| Compound muscle | 56.11 ± 22.74 | 31.77 ± 11.26 | 46.87 ± 15.54 |
| action potential | | | |
| (CMAP) (%) | | | |
| Maximum isometric | 54.89 ± 14.18 | 39.97 ± 13.82 | 51.35 ± 10.12 |
| tetanic force (ITF) (%) | | | |
| Wet muscle weight (%) | 67.11 ± 7.50 | 56.21 ± 10.39 | 56.63 ± 9.20 |
| Contracture angle (%) | 89.71 ± 8.01 | 76.15 ± 6.98 | 80.20 ± 6.49 |
| Axon density (/0.09 mm²) | 1056.45 ± 204.85 | 709.28 ± 139.04 | 1018.43 ± 160.49 |
| Myelin thickness (μm) | 1.25 ± 0.31 | 1.06 ± 0.23 | 1.22 ± 0.31 |
| Fascicular area (μm²) | 390313.95 ± 146399.04 | 258585.35 ± 103002.46 | 267691.82 ± 98925.87 |
| S110b (%) | 66.21 ± 7.02 | 48.01 ± 6.24 | 62.83 ± 6.84 |
| NFH-N52 (%) | 371.56 ± 45.11 | 325.14 ± 26.22 | 377.75 ± 31.87 |

16 Weeks

| | | | |
|---|---|---|---|
| Compound muscle | 58.31 ± 11.42 | 42.68 ± 10.44 | 55.75 ± 9.55 |
| action potential | | | |
| (CMAP) (%) | | | |
| Maximum isometric | 57.38 ± 11.68 | 44.89 ± 9.67 | 56.97 ± 9.91 |
| tetanic force (ITF) (%) | | | |
| Wet muscle weight (%) | 69.10 ± 8.10 | 63.84 ± 6.77 | 65.53 ± 6.81 |
| Contracture angle (%) | 88.84 ± 8.68 | 86.06 ± 3.43 | 88.51 ± 6.61 |
| Axon density (/0.09 mm²) | 1128.40 ± 279.72 | 838.92 ± 202.56 | 1110.17 ± 169.63 |
| Myelin thickness (μm) | 1.21 ± 0.29 | 0.94 ± 0.19 | 1.25 ± 0.26 |
| Fascicular area (μm²) | 393463.40 ± 133093.86 | 289121.28 ± 120666.28 | 359573.35 ± 359573.35 |
| S110b (%) | 68.76 ± 5.27 | 47.00 ± 5.04 | 70.24 ± 4.64 |
| NFH-N52 (%) | 408.72 ± 31.56 | 371.83 ± 31.73 | 441.97 ± 42.15 |

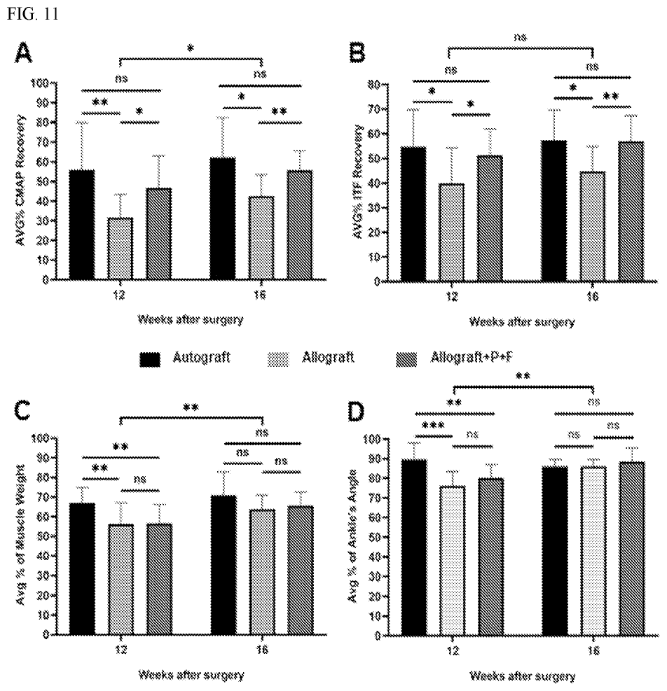

As showed in FIG. 11A, the percentage of CMAP recoveries at 16 weeks was significantly higher than that at 12 weeks in all three groups (p<0.05). In addition, the CMAP recoveries in the Allograft+PEP treatment group were superior to CMAP recoveries in allograft alone group at both 12 weeks and 16 weeks (p<0.05) and statistically insignificantly different than the autograft group at both 12 weeks and 16 weeks.

The effects of PEP-based therapy were further confirmed by the isometric tetanic force test. FIG. 11B shows there was a significant difference of average recovery rates between allografts with and without PEP−TISSEEL (p<0.05). Again, the difference between autograft and allograft+PEP+TISSEEL was statistically insignificant. These results suggest the recoveries of electrophysiological properties after allograft+PEP+TISSEEL treatment are comparable to those in the autograft treatment group.

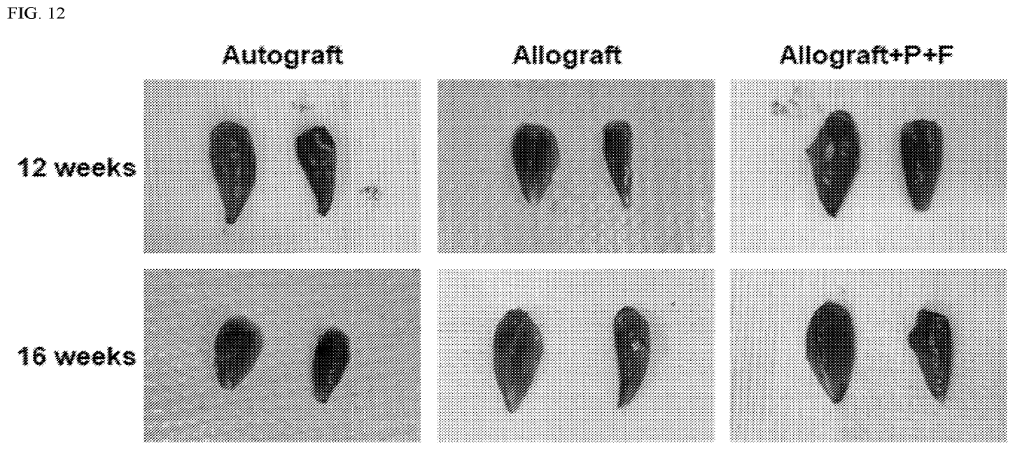

In addition to electrophysiology, the anterior tibialis muscle recovery (FIG. 11C) and ankle contracture angles (FIG. 11D) were also considered as another sign of peripheral nerve functional recovery. As shown in FIG. 12, the weights of anterior tibialis significantly increased from 12 weeks to 16 weeks in all three groups (p<0.01). The muscle weights were similar among all three treatment groups.

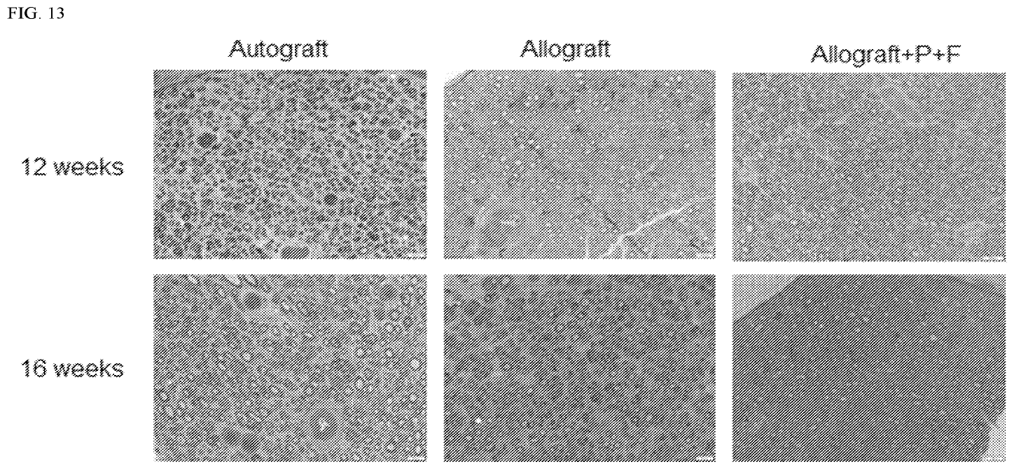

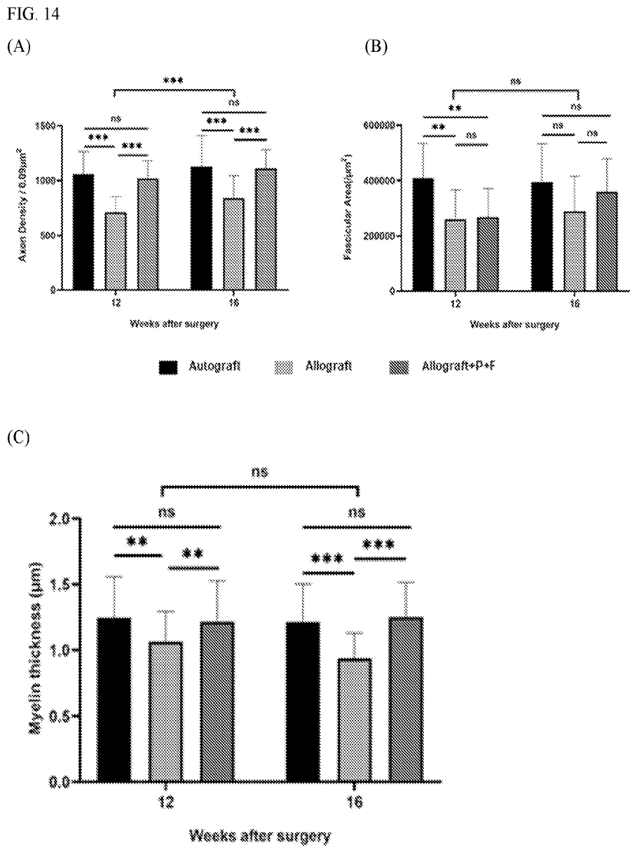

Morphometric analysis of rejuvenated nerves was completed to quantitatively measure density of axons, fascicular area, and myelin thickness at two different time points. FIG. 13 shows toluidine-blue-stained transverse slides of peroneal nerve were examined under light microscopy. FIG. 14A shows that axon density of allograft+PEP+TISSEEL group was significantly higher than the allograft group (p<0.001) and statistically comparable to the autograft group at both time points. Results at 16 weeks showed a positive increasing trend compared to those of 12 weeks in each group (p<0.001). Fascicular area (FIG. 14B) in autograft group was significantly greater than the allograft groups (with or without PEP+TISSEEL) at 12 weeks after surgery (p<0.01). No other significant difference was found between the three groups at 16 weeks. Myelin thickness (FIG. 14C) showed similar results with axon density. The outcomes in the allograft+PEP+TISSEEL group was significantly better than allograft treatment alone and comparable to the autograft group at both time points.

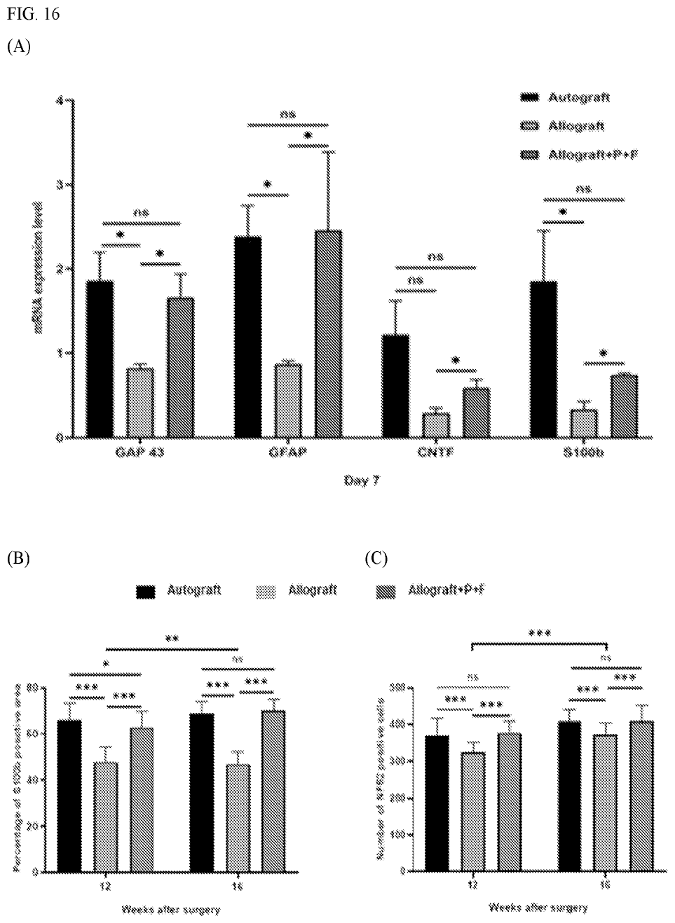

Neurotrophic factors are mediators of nerve regeneration processes. Samples taken from the regenerated nerves from each group were subjected to RT-PCR to quantify neurotrophic genes (CNTF and GFAP) and Schwann cell-related genes (S100 and GAP43) at seven days after post-surgery (FIG. 16A). Expression of GAP43, CNTF, GFAP, and S100b in the allograft+PEP+TISSEEL group were significantly increased compared to allograft alone group. (p<0.05).

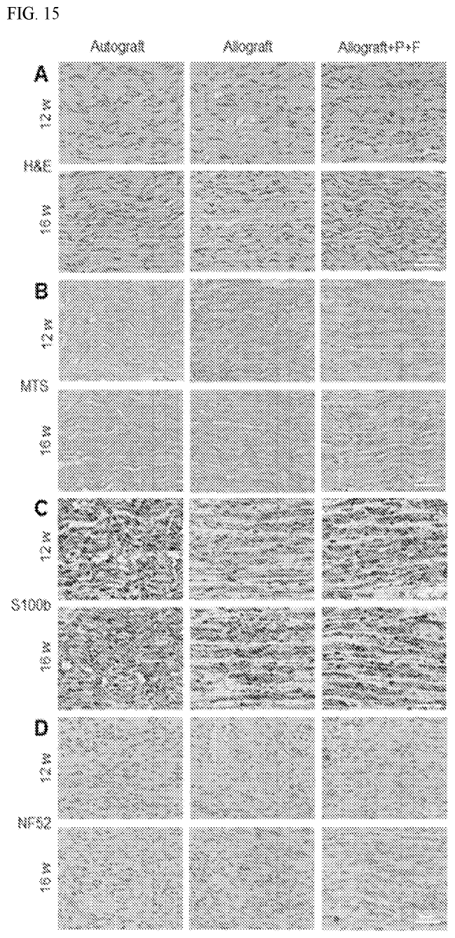

Hematoxylin-eosin (H&E) and Masson trichrome (MT) staining were used to evaluate the regenerated nerves of the longitudinal sections at 12 weeks and 16 weeks post-implantation. As presented in FIG. 15, newly formed axons with linearly ordered structures were observed growing into the grafts, Schwann cells proliferated significantly, nerve fibers showed mild swelling, and blood vessels had formed. H&E staining (FIG. 15A) also identified some inflammatory cells, containing macrophages and neutrophils, appeared around the Schwann cells. The samples from the autograft group and the allograft+PEP+TISSEEL group had more newly formed nerves and more correctly ordered linear guidance for growth compared with group treated with allograft alone. Masson trichrome staining (FIG. 15B) revealed that the quantity and order of regenerated nerve fibers in the allograft alone group were rare and disorganized compared to the autograft group and the allograft+PEP+TISSEEL group. S100beta staining was conducted to observe the regeneration of Schwann cells in the grafted nerves (FIG. 16C). The processed data (FIG. 16B) revealed significantly higher level of S100beta-positive areas in the autograft group and the and allograft+PEP+TISSEEL group compared to allograft only group at 12 weeks and 16 weeks post-implantation (p<0.01), consistent with the results in the number of NFH-N52-positive cells (p<0.05) (FIG. 16B).

In peripheral nerve repair, priority is given to the restoration of function. Recently, strategies using tissue engineered materials combined with cell-based therapy have attracted attention as a strategy for producing better functional recovery in cases of long nerve defects. Decellularized nerve allograft is one of these promising options and is available in a variety of length and diameters. Decellularized nerve allografts provide an extracellular matrix scaffold for axonal regrowth. However, deficiency of Schwann cells and neurotrophic factors within the allograft may result in inferior results compared to autograft. If recovery following allograft could be improved or made equivalent to autograft, allograft would become a more accepted therapeutic strategy for peripheral nerve gap repair since allografts are readily available, avoid having to locate donor sites in the patient, and decrease operative time. To improve functional results with nerve allograft, this study used PEP on decellularized allograft to imitate the indigenous microenvironment. As a result, allograft axon remodeling was made comparable to a nerve autograft.

In the current study, nerve allografts were treated with an exosome-based, cell-free, plasma-derived product (PEP) delivered with fibrin glue and used the combination to examine the effect of sciatic nerve regeneration. Decellularized nerve allograft with PEP-fibrin glue showed similar results as the reverse autograft, both of which were superior to allograft alone. The PEP-fibrin allografts exhibited better functional results than supplemented allografts after 12 weeks and 16 weeks. Electrophysiological function was verified by CMAP and ITF, which showed higher amplitude and force in all allograft+PEP+TISSEEL groups, indicating effective recovery of tibialis muscle after reinnervation. Histomorphometry showed axonal regrowth and re-myelination accompanying functional recovery in this model, as compared to fascicular area enhancement. RT-PCR and immunohistochemical (IHC) staining demonstrated that S100b, GAP43, CNTF, and GFAP were expressed at high level.

This disclosure therefore describes the use of human plasma-derived exosome in combination with decellularized nerve allograft for regenerating peripheral nervous tissue. The use of PEP in combination with allograft provides one or more of the following advantages. PEP is a readily-available off-the-shelf product. PEP provides high purity with less processing time than conventional exosome preparations. Reconstituting PEP does not require special equipment and can be performed in the operating room, saving time and lowering the risk of developing patient morbidity. Further, the allograft+PEP+fibrin cell-free reconstructive construct does not require immunosuppression.

Macroenvironmental factors, microenvironmental factors, and neurotrophic factors influence axonal regeneration. The data presented herein show that a human plasma-derived exosome product (PEP) improves the proliferation and migration of Schwann cells. Schwann cell migration, in particular, facilitates bio-regeneration. Histological evaluation and gene expression suggests that PEP-fibrin glue treatment provided allografts with growth-promoting and neuronal regulation cytokines for nerve regeneration. Taken together, these results indicated that the application of plasma-derived exosome delivery cell-free system can provide growth factors comparable to that of a cell-based system to improve peripheral nerve regeneration.

This disclosure therefore describes compositions and methods for improving repair of peripheral nervous tissue. Generally, the compositions include PEP and a pharmaceutically acceptable carrier. In a surgical setting, the PEP may be combined with a carrier that is suitable for application to peripheral nervous tissue such as, for example, a surgical glue or a tissue adhesive. The peripheral nervous tissue may be autologous or allogeneic.

Thus, the method includes administering an effective amount of the composition to peripheral nervous tissue in need of repair. In this aspect, an “effective amount” is an amount effective to increase maximal isometric tetanic force, accelerate motor functional recovery, accelerate axon maturation, increase axon diameter, and/or induce nerve regenerative gene expression compared to untreated peripheral nervous tissue or peripheral nervous tissue treated with carrier (no PEP) alone.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue or tissue adhesive.

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to peripheral nervous tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In some embodiments, the method can include administering sufficient PEP to provide a dose of, for example, from about a 0.01% solution to a 100% solution to the subject, although in some embodiments the methods may be performed by administering PEP in a dose outside this range. As used herein, a 100% solution of PEP refers to PEP solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In some embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In some embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In some embodiments, the method can include administering sufficient PEP to provide a dose characterized by a range having endpoints defined by any minimum dose identified above and any maximum dose that is greater than the minimum dose. For example, in some embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

Alternatively, a dose of PEP can be measured in terms of the PEP exosomes delivered in a dose. Thus, in some embodiments, the method can include administering sufficient PEP to provide a dose of, for example, from about 1×10⁶ PEP exosomes to about 1×10¹⁵ PEP exosomes to the subject, although in some embodiments the methods may be performed by administering PEP in a dose outside this range.

In some embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In some embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In some embodiments, the method can include administering sufficient PEP to provide a dose characterized by a range having endpoints defined by any minimum dose identified above and any maximum dose that is greater than the minimum dose. For example, in some embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

A single dose may be administered all at once, continuously for a prescribed period of time, or in multiple discrete administrations. When multiple administrations are used, the amount of each administration may be the same or different. For example, a prescribed daily dose may be administered as a single dose, continuously over 24 hours, or as two or more administrations, which may be equal or unequal. When multiple administrations are used to deliver a single dose, the interval between administrations may be the same or different. In certain embodiments, PEP may be administered from a one-time administration, for example, during a surgical procedure.

In certain embodiments in which multiple administrations of the PEP composition are administered to the subject, the PEP composition may be administered as needed to regenerate the peripheral nervous tissue to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In some embodiments, the method can include multiple administrations of PEP at an interval (for two administrations) or intervals (for more than two administrations) characterized by a range having endpoints defined by any minimum interval identified above and any maximum interval that is greater than the minimum interval. For example, in some embodiments, the method can include multiple administrations of PEP at an interval or intervals of from one day to six months such as, for example, from three days to ten days. In certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In some embodiments, the methods can include administering a cocktail of PEP that is prepared from a variety of cell types, each ceil type having a unique neuron-supporting profile—e.g. protein composition and/or gene expression. In this way, the PEP composition can provide a broader spectrum of neuron-supporting activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1—Autograft Model

### Mice

Ninety-six Sprague Dawley (SD) male rats weighing 250 g to 300 g were randomized to one of three groups. All groups had a 10-mm nerve defect created in the sciatic nerve and the nerve segment was reversed and used as an autograft. Group I was the control group, Group II had the addition of fibrin glue (TISSEEL, Baxter International Inc., Deerfield, Illinois) to the reconstruction, and Group III had PEP suspended in the fibrin glue. The left leg was used for all surgical procedures. The right leg was used as the control. After surgery, the rats were given food and water freely and housed two or three rats per cage with a twelve-hour light-dark cycle.

Animals were sacrificed to evaluate gene expression at three days (n=4), seven days (n=4), 12 weeks (n=4), and 16 weeks (n=4) post-surgery. Functional recovery was assessed by compound muscle action potential (CMAP), isometric tetanic force, and wet muscle mass of the tibialis anterior muscle at 12 weeks and 16 weeks post-surgery (n=24). Histology of the peroneal nerve segment distal to the graft was evaluated at 12 weeks and 16 weeks post-surgery (n=24).

Index Surgical procedure All animals were anesthetized by inhalation of isoflurane (Piramal Critical Care, Inc., Bethlehem, Pennsylvania). Enfloxacin (ENROSITE Injection 2.27%, Norbrook Laboratories Ltd., Newry, UK), ibuprofen, and BUPRENORPHINE SR (ZooPharm Pharmacy, Windsor, Colorado) were also administered before surgery. The left sciatic nerve was exposed. A 10-mm segment of the nerve was excised and reversed for use as an autograft, which was then repaired using 10-0 nylon epineural sutures (10-0 ETHILON BV75-3, Ethicon, Inc., Somerville, New Jersey) under an operating microscope (Foldable Tube; f170, Stand; Universal S3, Carl Zeiss, Oberkochen, Germany). After nerve repair, incised fascia was repaired, and skin was closed using 5-0 vicryl sutures (5-0 VICRYL RAPIDE, Ethicon, Inc., Somerville, New Jersey).

### Purified Exosome Product (PEP)

PEP (Rion LLC, Rochester MN) is a composition of leukocyte-depleted human exosomes that are circular to oval shaped with a size range of 120 nm to 140 nm. (Qi, 2020; International Patent Publication No. WO 2019/118817 A1). PEP was prepared in meeting current good manufacturing practice (GMP) standards as an off-white to light yellow powder stored at room temperature. Stability testing, which included validated GMP quality control assays that ensures robust impact on cell proliferation and angiogenesis, confirmed viability of PEP for over 12 months at room temperature. The size distribution and concentration of exosomes were measure by NANOSIGHT (Malvern, Worcestershire, UK) and analyzed by Nanoparticle Tracking Analysis Software (Malvern, Worcestershire, UK) according to the manufacturer's instructions.

PEP was aseptically processed, pyrogen-free and does not contain preservatives. All lots of PEP have been non-reactive to testing of Hepatitis B, Hepatitis C, Human immunodeficiency virus (HIV)-1, HIV-2, Human T-cell leukemia-lymphoma virus (HTLV)-I, HTLV-II, syphilis, West Nile Virus, Zika Virus, and Trypanosoma cruzi. Residual moisture for each PEP preparation is ≤8% and endotoxin values must be ≤0.5 EU/mL and final sterility cultures must show no growth. PEP powder needs to be dissolved in solution before use and dissolving one vial of PEP in 1 mL solution will be subsequently referred to as 100% PEP, with dilutions of this concentration being referred to as lower percentages of PEP solution.

Cytokine Characterization of PEP

PEP was characterized versus mesenchymal stem cells, as a well characterized regenerative medicine platform, to display enriched immunomodulation, antioxidant behavior, angiogenic potential, and mitogenic effects. The Proteome Profiler Human XL Cytokine Array Kit (ARY022B, R&D Systems, Abingdon, UK) was use for semi-quantitative determination of the enrichment of 105 cytokines, chemokines, growth factors, angiogenesis markers, and other soluble proteins in PEP compared to mesenchymal stem cell conditioned media. Conditioned media (CM) was collected from adipose derived mesenchymal stem cells (AMSC) after two days in culture with reduced serum media. The cytokine array analysis was performed according to the manufacturer's instructions using 50 μg of protein. Protein was quantified using a BCA Protein Assay Kit (Pierce, Thermo Fisher Scientific, Waltham, Massachusetts). After film exposure of 15 minutes, the intensity of average signal (pixel density) was quantified using Quick Spots HLImage++software (Western Vision Software, Salt Lake City, Utah).

Fold change between PEP conditioned media and AMSC conditioned media was calculated and cytokine array factors were ranked. STRING Database (Snel et al., 2000. Nucleic Acids Res 28(18):3442-3444; Szklarcyk et al., 2019. Nucleic Acids Res 47:D607-613) was used to perform network analysis identify potential enriched interaction. Gene ontology functional enrichment analysis on the top 10-fold upregulated factors was assessed to identify key pathways up-regulated in PEP.

### PEP Antioxidant Western Blot

Three different batches of PEP were dissolved into a 20% solution (5 mL saline in PEP vial), filtered with a 0.2-micron filter, and the concentration of proteins was quantified using a BCA Assay Kit (Pierce, Thermo Fisher Scientific, Waltham, Massachusetts). From this, 20 μg of protein from each sample was lysed, prepared in 1× Laemmli Sample Buffer (Bio-Rad Laboratories, Inc., Hercules, CA), and heated at 85° C. for three minutes. Samples were run on a 12.5% precast gel (CRITERION, Bio-Rad Laboratories, Inc., Hercules, CA) and transferred onto PVDF membrane. The membrane was blocked in 5% milk in Tris-buffered 0.1% Tween 20. Primary antibodies used included overnight incubation at 4° C. with anti-CD63 (ab59479, Abcam, Cambridge, UK), anti-Superoxide Dismutase-1 (cat. #2770, Cell Signaling Technology, Inc., Danvers, Massachusetts) and anti-Heme Oxygenase-1 (cat. #374090, Millipore Sigma, Burlington, MA). Secondary antibody was horseradish peroxidase conjugated (Jackson ImmunoResearch Laboratories, Inc., West Grove, PA). Membranes were developed using SuperSignal Pico PLUS (Thermo Fisher Scientific, Inc., Waltham, MA).

iPSC-Neuron Oxidative Stress

Human IPSC-derived mixed neurons were seeded at a density of 50,000 cells/cm² in a 96—well plate. Cells were grown in Neurobasal medium (Thermo Fisher Scientific, Inc., Waltham, MA) supplemented with 1× final concentration of B-27 Supplement, minus antioxidants (Thermo Fisher Scientific, Inc., Waltham, MA). Cells were incubated with 20% PEP, 10% PEP, 5% PEP, or no PEP dissolved in the culture media described above for 16 hours. Media in the wells was then exchanged for the basal medium containing no PEP and LY83583 (Millipore Sigma, Burlington, MA) at 20 μM final concentration. INCUCYTE Caspase-3/7 Green Apoptosis Assay Reagent (Essen BioScience, Inc., Ann Arbor, MI) was added to each well to detect apoptotic cells. INCUCYTE NucLight Rapid Red Reagent was added to each well to detect cell nuclei. Apoptotic cells and cell nuclei were detected using a lice cell analysis system (INCUCYTE, Essen BioScience, Inc., Ann Arbor, MI).

### Reconstitution of Fibrin Glue and PEP-Fibrin Glue

TISSEEL (Baxter International Inc., Deerfield, IL) fibrin sealant was prepared according to the manufacturer's instructions for a final total of 2 mL. Of this glue mixture, 0.5 mL was used in Group II by applying the glue around the reverse autograft nerve. 1 mL of TISSEEL fibrinolysis inhibitor solution was added to 1 vial of PEP to obtain a 1:1 reconstitution (100%) according to the manufacturer's instructions. Following reconstitution, 100 μL of the PEP-fibrinolysis solution was then added to 900 μL of the fibrinolysis inhibitor to achieve a 10% PEP solution. The remaining steps were then conducted according to the TISSEEL instructions for use. Briefly, the 10% PEP solution was added to the sealer protein concentrate powder to yield 1 mL final solution. Calcium chloride solution was added to thrombin to also yield a 1 mL solution. These solutions were then drawn into the supplied syringes and applied to the site of interest for a final 5% PEP−TISSEEL solution.

### Outcome Measures (Survival Procedures)

### Compound Muscle Action Potential

Compound Muscle Action Potential (CMAP) analysis was performed as previously described (Giusti et al., 2012. JBone Joint SurgAm 94(5):410-417). All animals were anesthetized by intraperitoneal injection of ketamine (KETASET, Zoetis Services LLC, Parsippany, NJ) and xylazine. The sciatic nerve was exposed and the CMAP of the tibialis anterior muscle was measured using a bipolar electrode clamped proximal to the graft site (n=24 in each group). CMAP results were normalized to the contralateral side.

### Isometric Tetanic Force

Maximum isometric tetanic force measurements of the tibialis anterior muscle were performed at 12 weeks (n=12 in each group) and 16 weeks (n=12 in each group) after surgery as previously described (Shin et al., 2008. Microsurgery 28(6):452-457). This validated method provides a reproducible quantitative evaluation of motor recovery. Results were expressed as a percentage compared to the contralateral, unoperated side.

### Outcome Measures (Non-Survival Procedures)

### Muscle Wet Weight

After the isometric tetanic force testing, the animals were sacrificed by an overdose of pentobarbital. The tibialis anterior muscles were carefully removed bilaterally (n=24 in each group) and the wet muscle weight obtained. Results were expressed as a percentage compared to the contralateral, unoperated side.

### Neurogenesis Gene Expression Analysis

Total RNA was isolated from harvested whole sciatic nerve (graft, 3 mm proximal, and 3 mm distal from nerve repair sites) and dorsal root ganglion (L4-6 level) (n=8 in each group) using Trizol reagent (Invitrogen, Life Technologies, Grand Island, New York). RNA was quantitated using a with a DS-11 spectrophotometer (DeNovix, Inc., Wilmington, DE). cDNA was synthesized from RNA obtained from the sciatic nerve and dorsal root ganglion of Group I mice and Group III mice at three days post-surgery and seven days post-surgery. cDNA was synthesized from RNA using theISCRIPT cDNA Synthesis Kit (Bio-Rad Laboratories, Inc., Hercules, CA) as previously described (Baglio et al., 2012. Front Physiol 3:359). The cDNA was subjected to RT² PROFILER PCR Array (Qiagen, Hilden, 24ermany) analysis (n=3 each) as previously described (Lai et al., 2012. Front Physiol 3:228).

The geomean of beta-actin, beta-2 micro-globulin, hypoxanthine phosphoribosyl transferase 1, lactate dehydrogenase, and ribosomal protein large P1 was used as the reference for gene expression. The gene expression at Ct value <35, and fold regulation <−1.5 or >1.5 were analyzed for statistical significance.

### Quantitative Real-Time PCR Analysis

qRT-PCR was performed in triplicate using a real-time PCR detection system (ICYCLER, Bio-Rad Laboratories, Inc., Hercules, CA) and a PERFECTA SYBR Green Fast Mix for iQ real-time PCR kit (Quantabio, Beverly, MA) as previously described (Baglio et al., 2012. Front Physiol 3:359; Lai et al., 2012. Front Physiol 3:228). The PCR results were calculated from the Ct and normalized using the geomean of glyceraldehyde-3-phosphate dehydrogenase and beta-actin. Rat-specific primers were used for growth-associated protein 43 (GAP43), S100 calcium-binding protein beta (S100b), nerve growth factor (NGF), and vascular endothelial growth factor A (VEGFA). Primers were designed with Primer3 software (Untergasser et al., 2012. Nucelic Acids Res 40(15):e115; Koressaar T and Remm M, 2007. Bioinformatics 23(10):1289-1291; Koressaar et al., 20118. Bioinformatics 34(11):1937-1938) and purchased from Integrated DNA Technologies (Coralville, Iowa). Results were normalized to the Group I data.

### Histomorphometry

Segments of the peroneal nerve distal to the autograft were harvested and prepared for histomorphometric measurements as described previously (Kim et al., 2018. Microsurgery 38(1):66-75). Five-millimeter sections of the nerves were fixed in 2% Trump's solution. Specimens were then embedded in resin, cut transversely and stained with Toluidine Blue. Images were analyzed using ImageJ 1.52s (Schneider et al., 2012. Nature Methods 9(7):671-675). Axon density (the number of myelinated axons/field; 0.036 mm²) and average axonal diameter (m) were measured at 400× magnification and analyzed in two random fields (Katsuda et al., 2013. Proteomics 13(10-11):1637-1653). All histomorphometric measurements were performed by two blinded researchers.

### Statistical Analysis

The number of rats required in each group was determined by the results of previous studies on primary outcome, isometric tetanic force (Shin et al., 2008. Microsurgery 28(6):452-457; Kim et al., 2018, Microsurgery 38(1):66-75). Based on this data, 18 rats in each group would provide 80% power to detect a 10% difference between groups (α=0.05, two-sided). Eight additional rats per group were used in evaluation of gene expression by qRT-PCR. To compensate for potential attrition, the sample size was increased to 32 per group.

All statistical procedures were carried out using JMP 14.1.0 (SAS Institute Inc., Cary, NC). Data was expressed as mean values±standard error. Statistical analysis was performed by one-way analysis of variance (ANOVA). Intergroup differences were examined by the Tukey-Kramer post-hoc test. 2^{-ΔCt} values in PCR array neurogenesis were compared between the control Group I and PEP Group III using a two-tailed, unpaired Student's t-test. Values of p<0.05 were considered statistically significant.

### Example 2—Allograft Model

### Composition of PEP and its Delivery Scaffold

PEP was prepared and provided as described in Example 1.

TISSEEL (Baxter International Inc., Deerfield, IL) fibrin sealant was prepared according to the manufacturer's instructions for a final total of 2 mL. 1 mL of TISSEEL fibrinolysis inhibitor solution was added to 1 vial of PEP to obtain a 1:1 reconstitution (100%) solution. 100 μL of the PEP-fibrinolysis solution was blended into 900 μL of the fibrinolysis inhibitor to achieve a 10% PEP solution. The remaining steps were then conducted according to the TISSEEL instructions for use. Briefly, the 10% PEP solution was added to the sealer protein concentrate powder to yield 1 mL final solution. Calcium chloride solution was added to thrombin to also yield a 1 mL solution. These solutions were then drawn into the supplied syringes and applied to the site of interest for a final 5% PEP−TISSEEL solution.

### Preparation of Nerve Allograft

Thirty-two Sprague-Dawley (SD) male rats (276 g to 316 g) were enrolled into the donor group for nerve harvesting, which are heterogenic from Lewis rats that were used for nerve allograft. Bilateral sciatic nerves were transected to segments (17 mm-45 mm), any connective fascia or fat was removed prior to storage in a tube and bathed in phosphate buffered saline (PBS). To standardize the nerve decellularization procedures and produce nerve allografts that are similar to those clinically available, the harvested nerves were decellularized by Axogen Corp. (Alachua, FL). After decellularization, each graft was frozen for storage. For use during surgery, each graft was tailored to 10 mm in length and allowed to thaw in saline for 20 minutes at room temperature.

### Cell Culture

The RSC96 rat Schwann cell line was obtained from ATCC (American Type Culture Collection, Manassas, VA) and cultured with the solution composed of Dulbecco's modified Eagle's medium (DMEM, American Type Culture Collection, Manassas, VA), 10% fetal bovine serum (FBS, Neuromics, Edina, MN) and 1% Antibiotic-Antimycotic (AA, Sigma-Aldrich, St. Louis, MO) at 37° C. containing 5% CO₂.

### Cell Viability Assay

The Cell Counting Kit-8 (CCK-8, Dojindo Molecular Technologies, Inc., Rockville, MD) tests were applied to measure Schwann cell proliferation following the instructions provided by the manufacturer. Schwann cells were seeded at a density of 1×10⁴ cells per well of a 96-well plate with different doses 0% PEP, 5% PEP, 10% PEP, or 20% PEP. After cell adhesion, the cells were incubated in 100 μL DMEM-CCK-8 medium (DMEM: CCK-8=9:1, v v) under different doses of PEP for two hours at room temperature. Optical density at 490 nm was taken every 24 hours using a microplate reader (FLUOSTAR Omega, BMG Labtech, Offenburg, Germany).

### Cell Migration Assay

Schwann cells and different doses of PEP−TISSEEL were cocultured using a 24-well TRANSWELL system (0.4 μm, Corning, Inc., Corning, NY). First, 0.5 ml of the Schwann cell mixture with 0.5×10⁵ cells/ml were seeded on the lower compartment, and the PEP−TISSEEL was added in the upper compartment gently. After the cells reached 80-90% confluence, cell scratch experiments were performed using 200 μL pipette tips. All plates were scanned after 36 hours in the INCUCYTE S3 live cell analysis system (Essen BioScience, Inc., Ann Arbor, MI). The closure rate of scratch was evaluated using ImageJ software (Schneider et al., 2012. Nature Methods 9(7):671-675).

### Cell Apoptosis Assay

Cell apoptosis test was performed using the Annexin V-FITC/PI apoptosis detection kit (Molecular Probes, Inc., Eugene, OR). Different concentration of PEP−TISSEEL and 24-well TRANSWELL system (Corning, Inc., Corning, NY) was prepared as described above. Briefly, 2 μL Annexin V-FITC and 2 μL propidium iodide were added to the medium and placed at 37° C. for 20 minutes. The INCUCYTE analysis system (Essen BioScience, Inc., Ann Arbor, MI) was used to count mean confluence from nine non-overlapping images per well. The data was extracted by the INCUCYTE ZOOM software (Essen BioScience, Inc., Ann Arbor, MI) to estimate the total number of dead cells of each well.

### Cellular Uptake of PEP

PEP exosomes were marked with VYBRANT CM-Dil solution (Invitrogen, Carlsbad, CA). Schwann cells were co-cultured with 5% PEP on 8-wells LAB-TEK II chamber slide system (Thermo Fisher Scientific, Inc., Waltham, MA) for three days. After fixed with 4% paraformaldehyde solution and blocked with 5% bovine serum albumin (BSA), cells were co-cultured with the primary antibody (Rabbit Anti-S100 beta, Abcam52642, Cambridge, UK) and the secondary antibody (488 AffiniPure Goat Anti-Rabbit IgG, Jackson ImmunoResearch Laboratories, Inc., West Grove, PA). The nuclei were stained with Hoechst 33342 (Thermo Fisher Scientific, Inc., Waltham, MA). Targeted images were acquired using a confocal laser scanning microscopy (LSM 780, Zeiss Microscopy, Jena, Germany) at 594 nm.

### PEP Release Test

A nanoparticle tracking system (NS300, NanoSight Ltd., Malvern, UK) was used to measure the concentration and size range of PEP particles. Eight tubes of 10 ml phosphate buffered saline (PBS) were prepared. Then, 5% PEP−TISSEEL was added to the first seven tubes sequentially every 24 hours for seven days, then skipped to the last tube at the 14^{th} day. A 1-ml sample from each tube was loaded into the sample chamber of the nanoparticle tracking system and injected automatically. Videos (30 seconds) were captured and analyzed by the NanoSight NTA 3.2 software (NanoSight Ltd., Malvern, UK). The concentration and size range of PEP particles were reported as the mean±standard deviation (SD).

### Scanning Electron Microscopy (SEM)

Scanning electron microscopy was used to examine how PEP interacts with the surface of nerve allograft. Briefly, samples were fixed with Trumps solution at 4° C. overnight, then washed with phosphate buffered saline and dehydrated with ethanol. Critical point drying was achieved with carbon dioxide and the samples were coated with gold-palladium. The samples were visualized under a scanning electron microscope (Hitachi S-4700, Michigan Technological University, Houghton, MI, USA) with an acceleration voltage set at 5 kV.

### Surgical Procedure of Lewis' Rat Sciatic Nerve

The effects of PEP-loaded nerve allograft on axonal restoration were evaluated making use of a nerve defect animal model. Eighty-four adult male Lewis rats (198 g to 250 g, Charles River Laboratories, Inc., Wilmington, MA) were anesthetized via inhalation of isoflurane (Sigma-Aldrich, St. Louis, MO). The operative fields were draped with sterile towels. The sciatic nerve on the left side was exposed at mid-thigh via a gluteal muscle-splitting approach, and a 10-mm nerve was transected by a clean cut under a dissecting microscope. Three different treatments were applied to bridge the defect: autograft (ATG), allograft (ALG), and (3) allograft with PEP−TISSEEL (ALG+P+F). Both ends of the graft were anastomosed to the proximal and distal stumps with microepineurial suture, using 10-0 nylon sutures (ETHILON, Ethicon, Inc., Somerville, NJ). The wound was closed in layers with 5-0 vicryl sutures (Ethicon, Inc., Somerville) and application of antibiotic gel. All the rats were observed closely during the initial two hours recovery period and returned to housing in a central animal care facility. All animals were screened postoperatively for infections, weight loss, and any other disabilities.

### Quantitative RT-PCR Analysis

Rat sciatic nerve graft samples were harvested from all three groups of animals on postoperative Day 7. RNA was extracted using TRIZOL (Invitrogen Life Technologies, Grand Island, NY) and reversed transcribe into cDNA with the iScriptTM cDNA synthesis kit (Bio-Rad Laboratories, Inc., Hercules, CA). RT-PCR was conducted using a PerfeCTaTM SYBR Green Fast MixTM (Quantabio, Beverly, MA) on a C1000 TOUCH thermal cycler (Bio-Rad Laboratories, Inc., Hercules, CA). All primers were designed with Primer3 software and acquired from Integrated DNA Technologies (Coralville, Iowa). Rat-specific primers were used for growth-associated protein 43 (GAP43), ciliary neurotrophic factor (CNTF), S100 calcium-binding protein beta (S100b), and glial fibrillary acidic protein (GFAP). Results of triplicate samples were analyzed using the 2^{-ΔΔCT} method with GAPDH as the reference gene.

### Motor Functional Analysis

12 weeks and 16 weeks after surgery, functional measurements were performed after general anesthesia, with the healthy right legs being used as controls. Motor functionalities were evaluated using the angle of ankle contracture test, compound muscle action potential (CMAP) test, maximum isometric tetanic force (ITF) measurement, and measurement of the weight of tibialis anterior bilaterally. Histologic sections were also collected transversely from peroneal nerve and later used for histomorphometric analysis. Longitudinal sections were stained with hematoxylin and eosin (H&E), Masson trichrome staining, and immunohistochemistry staining. Details of these tests are described below:

Ankle Contracture Angle

Before the biomechanical tests, the angle of ankle contracture was measured and normalized to the non-operated side. This was done by positioning the ankle at its maximal passive plantar flexion, then measuring the angle formed by the tibial shaft and the dorsum of the foot.

Compound Muscle Action Potentials (CMAP)

CMAP procedures were similar to those previously described (Shin et al., 2008, Microsurgery 28(6):452-457). Briefly, after anesthesia using ketamine (KETASET, Fort Dodge Animal Health, Fort Dodge, Iowa) and xylazine, rats were fixed onto the testing block in a semi prone position. The sciatic nerve on the repaired side was re-exposed. A miniature stimulating electrode (Harvard Apparatus, Holliston, MA) was hooked around the nerve proximal to the graft. Bipolar electrodes (SD9; Grass Instrument Company, West Warwick, RI) were inserted into the belly of tibialis anterior exposed by skin incision in front of the shank. CMAPs were evaluated in the tibialis anterior using a NICOLET VIKINGQUEST portable electromyography (EMG) system (Natus Medical, Inc., Pleasanton, CA). Stimulation (duration: 0.02 ms, frequency: 0.5 Hz, stimulation amplitude: 2.7 mA) was used to generate a CMAP signal. CMAP data were normalized by using the contralateral side regarded as normal.

Maximum Isometric Tetanic Force (ITF)

Following the CMAP test, the incision extended distally to the peroneal branch was exposed to evaluate isometric tetanic force (ITF). The tibialis anterior tendon was then dissected distal to the ankle joint. The left hind limb was fastened to a wooden block on the platform with two Kirschner wires (K-wire; Stryker Orthopaedics, Kalamazoo, MI) respectively penetrated through knee and ankle joint. Peroneal nerve was connected to the same bipolar stimulating electrode as CMAP test. A force transducer (Model GM, Honeywell Inc., Columbus, OH) was attached to the distal end of tibialis anterior tendon using a clamp. Signals from the force transducer were processed by an amplifier and recorded using LabVIEW software (National Instruments Corp., Austin, TX). Parameter test was assessed with frequency 10 Hz, voltage 2V, duration 0.2 ms. For each measurement, supramaximal voltage was applied to ensure activation of all motor units in the muscle. The maximum isometric tetanic force was determined from the force-frequency curve that has the highest plateau. The same measurement was repeated for the contralateral side as well.

Weight of Tibialis Anterior Muscle

Rats were sacrificed after the biomechanical tests using an overdose of pentobarbital (Vortech Pharmaceuticals, Ltd., Dearborn, MI). Tibialis anterior without tendon was carefully harvested and weighed. Muscle weight was normalized to the healthy side.

Histomorphometry

After the mechanical evaluations, 3 mm of the peroneal nerve was obtained from its origin after branching off from the sciatic nerve and fixed in 2% Trump's solution. After dehydrated, the peroneal nerve segment was then embedded in paraffin embedding media. Samples were sectioned transversely into 5 m-thick slices and stained with 1% toluidine blue. All slides were then captured with a camera connected to a light microscope. Low magnification (10×) images were used to evaluate the total region in the regenerated nerve. Five non-overlapping areas per slice were randomly selected. Morphometric evaluations in the high-magnification (40×) images were captured. Axon cross sections were analyzed as previously described (Tang et al., 2019, JBone Joint SurgAm 101(10):e42). The density of axons, fascicular area and myelin thickness were evaluated using ImageJ software (Schneider et al., 2012. Nature Methods 9(7):671-675).

Immunohistochemistry

A 10-mm sciatic nerve sample was harvested from each nerve repair for subsequent analysis. The samples were collected and stored in 4% paraformaldehyde at −4° C. After embedded in paraffin, 5-m-thick longitudinal sections were sliced using a microtome and stained with hematoxylin and eosin (H&E) and Masson trichrome reagents. In addition, the axon and myelin sheath were labeled with NFH-N52 (Anti-Hypophosphorylated Neurofilament H antibody [N52] Abcam82259, Cambridge, UK) and S100beta (Rabbit Anti-S100 beta, Abcam52642, Cambridge, UK). The quantification of NFH-N52 and S100beta positive areas was evaluated with three non-overlapping randomly areas per specimen.

Statistics

All numerical data were reported as means±SEM or ±SD as indicated. Comparisons between two groups were conducted by the Student's t test, and comparisons among groups of more than two was done using one-way analysis of variance (ANOVA). All statistical analyses were done using GraphPad Prism statistical software (version 5, GraphPad Software, Inc, San Diego, CA). Results were statistically significant when p<0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
PEP; and
a pharmaceutically acceptable carrier comprising a surgical glue or tissue adhesive.

2. The composition of claim 1, wherein the PEP comprises spherical or spheroid exosomes having a diameter no greater than 300 nm.

3. The composition of claim 1, wherein the PEP comprises spherical or spheroid exosomes having a mean diameter of 110 nm±90 nm.

4. The composition of claim 3, wherein the PEP comprises spherical or spheroid exosomes having a mean diameter of 110 nm±50 nm.

5. The composition of claim 4, wherein the PEP comprises spherical or spheroid exosomes having a mean diameter of 110 nm±30 nm.

6. The purified exosome product of claim 1, wherein the PEP comprises:
from 1% to 20% CD63⁻ exosomes; and
from 80% to 99% CD63⁺ exosomes.

7. The purified exosome product of claim 1, wherein the PEP comprises at least 50% CD63⁻ exosomes.

8. The purified exosomes product of claim 1, wherein the PEP comprises from 1×10¹¹ PEP exosomes to 1×10¹³ PEP exosomes.

9. The purified exosome product of claim 8, wherein the PEP comprises from 1×10¹² PEP exosomes to 1×10¹³ PEP exosomes.

10. A method of promoting growth of peripheral nervous tissue, the method comprising applying the composition of claim 1 to injured peripheral nervous tissue.

11. The method of claim 10, wherein the peripheral nervous tissue is an autograft.

12. The method of claim 10, wherein the peripheral nervous tissue is an allograft.
